(19) United States
(12) Patent Application Publication
(10) Pub. No.: US 2019/0209517 A1
(43) Pub. Date: Jul. 11, 2019

(54) METHODS OF STABILIZING DRONABINOL

(71) Applicant: Insys Development Company, Inc., Chandler, AZ (US)

(72) Inventors: Eshwaran Narayanan, Chandler, AZ (US); Rajesh Wakaskar, Chandler, AZ (US); Rachana Inavolu, Chandler, AZ (US); Thrimoorthy Potta, Phoenix, AZ (US); Venkat R. Goskonda, Phoenix, AZ (US)

(21) Appl. No.: 16/242,098

(22) Filed: Jan. 8, 2019

Related U.S. Application Data
(60) Provisional application No. 62/615,488, filed on Jan. 10, 2018.

(51) Int. Cl. A61K 31/352; A61K 9/00; A61K 47/10; A61K 47/14; A61K 47/18; A61J 1/00

(52) U.S. Cl. CPC A61K 31/352; A61K 9/0053; A61J 1/00; A61K 47/14; A61K 47/183; A61K 47/10

(57) ABSTRACT

The present invention is directed to methods of stabilizing an oral pharmaceutical composition comprising dronabinol containing the steps filling a container, capping the container and placing the capped container in a secondary packaging system.

### BACKGROUND OF THE INVENTION

Delta-9-tetrahydrocannabinol (also known as THC, dronabinol and Δ9-THC) is a naturally occurring compound and is the primary active ingredient in the controlled substance marijuana. Marijuana refers to the dried flowers and leaves of Cannabis Sativa, the hemp plant. These parts of the plant contain several compounds called cannabinoids (including dronabinol), that may help patients with certain disease conditions.

Currently, dronabinol is commercially available in the U.S. as a sesame oil solution in a soft gelatin capsule under the tradename Marinol® from AbbVie, Inc., which is orally administered. Upon oral administration, the gelatin dissolves, releasing the drug. The dronabinol dissolved in sesame oil is then absorbed during its passage through the gastrointestinal tract. The Marinol® soft gelatin capsule form of dronabinol is highly unstable at room temperature, and it is required that the product be stored at refrigerated (2-8° C.) or cool (8-15° C.) conditions (Marinol® package label, Physicians' Desk Reference®, ed. 2003). Additionally, Marinol® should be packaged in a well-closed container.

The need to store dronabinol product in a refrigerator is a major disadvantage for a pharmaceutical product. Accordingly, there is a need for developing a room temperature stable dronabinol product that addresses problems associated with the storage of a dronabinol at refrigerated conditions and for patient convenience.

### SUMMARY OF THE INVENTION

The present invention is directed to a method of stabilizing an oral pharmaceutical composition comprising:
from about 0.1% to about 5% w/w dronabinol; and
optionally, from about 20% to about 40% w/w water, from about 15% to about 65% w/w of ethanol, from about 2% to about 10% w/w propylene glycol, from about 8% to about 20% w/w polyethylene glycol, and an excipient selected from the group consisting of butylated hydroxyanisole (“BHA”), butylated hydroxytoluene (“BHT”), disodium ethylenediaminetetraacetic acid (“EDTA”), a paraben and a combination thereof,
   comprising the steps of:
a. filling a container with the oral pharmaceutical composition under vacuum or a gas overlay;
b. capping the container to create a gaseous headspace in the container;
c. placing the container in a secondary packaging system with or without a gas overlay; with or without an oxygen absorbing means; and with or without an oxygen indicator;
d. sealing the secondary packaging system,
   wherein the gaseous headspace contains less than 20% oxygen and wherein the secondary packaging system is selected from the group consisting of a pouch and a blister package.

The present invention is directed to a method of stabilizing an oral pharmaceutical composition comprising:
from about 0.1% to about 5% w/w dronabinol; and
optionally, from about 20% to about 40% w/w water, from about 15% to about 65% w/w of ethanol, from about 2% to about 10% w/w propylene glycol, from about 8% to about 20% w/w polyethylene glycol, and an excipient selected from the group consisting of butylated hydroxyanisole (“BHA”), butylated hydroxytoluene (“BHT”), disodium ethylenediaminetetraacetic acid (“EDTA”), a paraben and a combination thereof,
   comprising the steps of:
a. filling a glass bottle with the oral pharmaceutical composition under a vacuum;
b. capping the glass bottle to create a gaseous headspace in the glass bottle;
c. placing the glass bottle in a secondary packaging system with or without a gas overlay; with or without an oxygen absorbing means; and with or without an oxygen indicator;
d. sealing the secondary packaging system,
   wherein the gaseous headspace contains less than 20% oxygen and wherein the secondary packaging system is selected from the group consisting of a pouch and a blister package.

The present invention is directed to a method of stabilizing an oral pharmaceutical composition comprising:
from about 0.1% to about 5% w/w dronabinol; and
optionally, from about 20% to about 40% w/w water, from about 15% to about 65% w/w of ethanol, from about 2% to about 10% w/w propylene glycol, from about 8% to about 20% w/w polyethylene glycol, and an excipient selected from the group consisting of butylated hydroxyanisole (“BHA”), butylated hydroxytoluene (“BHT”), disodium ethylenediaminetetraacetic acid (“EDTA”), a paraben and a combination thereof,
   comprising the steps of:
a. filling a glass bottle with the oral pharmaceutical composition under a gas overlay;
b. capping the glass bottle to create a gaseous headspace in the glass bottle;
c. placing the glass bottle in a secondary packaging system with or without a gas overlay, with or without an oxygen absorbing means; and with or without an oxygen indicator;
d. sealing the secondary packaging system,
   wherein the gaseous headspace contains less than 20% oxygen and wherein the secondary packaging system is selected from the group consisting of a pouch and a blister package.

The present invention is further directed to a container comprising an oral pharmaceutical composition comprising from about 0.1% to about 5% w/w dronabinol, wherein the container further comprises a gaseous headspace containing less than 20% oxygen.

### DETAILED DESCRIPTION OF THE INVENTION

The present invention provides room temperature stable dronabinol compositions through novel packaging methods.

As used herein, the term “dronabinol” refers to the cannabinoid delta-9-tetrahydrocannabinol having the CAS number 1972-08-03 and the following chemical structure

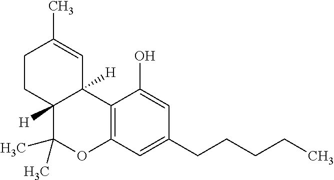 [SMILES: CCCCCc1cc(O)c2c(c1)OC(C)(C)[C@@H]1CCC(C)=C[C@@H]21]

Methods of the present invention may further be used to provide room temperature stable compositions containing any cannabinoid. The term “cannabinoid”, as used herein, includes natural, synthetic and semi-synthetic cannabinoids. Semi-synthetic cannabinoids include non-natural derivatives of cannabinoids which can be obtained by derivatization of natural cannabinoids and which are unstable like natural cannabinoids.

The cannabinoid may be included in its free form or in the following forms: a salt; an acid addition salt of an ester; an amide; an enantiomer; an isomer; a tautomer; a prodrug; a derivative of an active agent of the present invention; different isomeric forms, including, but not limited to enantiomers and diastereoisomers, both in pure form and in admixture, including racemic mixtures; and enols. The term “cannabinoid” is also meant to encompass derivatives that are produced from another compound of similar structure by the replacement of one atom, molecule or group by another. Cannabinoids that may be stabilized by methods of the present invention, include, but is not limited to, delta-8-tetrahydrocannabinol, delta-9-tetrahydrocannabinol, cannabidiol, cannabinol, cannabigerol, nabilone, delta-9-tetrahydro cannabinotic acid, the nonpsychotropic cannabinoid 3-dimethylnepty II carboxylic acid homologine 8, delta-8-tetrahydrocannabinol (1. Med. Chem. 35, 3135, 1992), prodrugs of cannabinoids, and pharmaceutically acceptable salts and complexes of cannabinoids.

In a most preferred embodiment, the cannabinoid is dronabinol.

As used herein, all numerical values relating to amounts, weights, and the like, that are defined as “about” each particular value is plus or minus 10% of the particular value. For example, the phrase “about 10% w/w” is to be understood as “9% w/w to 11% w/w.” Therefore, amounts within 10% of the claimed value are encompassed by the scope of the claims.

All weights herein refer to % w/w or percent weight of the total composition.

The present invention is directed to a method of stabilizing an oral pharmaceutical composition comprising:
from about 0.1% to about 5% w/w dronabinol; and
optionally, from about 20% to about 40% w/w water, from about 15% to about 65% w/w of ethanol, from about 2% to about 10% w/w propylene glycol, from about 8% to about 20% w/w polyethylene glycol, and an excipient selected from the group consisting of butylated hydroxyanisole (“BHA”), butylated hydroxytoluene (“BHT”), disodium ethylenediaminetetraacetic acid (“EDTA”), a paraben and a combination thereof,
   comprising the steps of:
a. filling a container with the oral pharmaceutical composition under a vacuum or a gas overlay;
b. capping the container to create a gaseous headspace in the container;
c. placing the container in a secondary packaging system with or without a gas overlay, with or without an oxygen absorbing means; and with or without an oxygen indicator;
d. sealing the secondary packaging system,
   wherein the gaseous headspace contains less than about 20% oxygen, preferably less than about 10% oxygen and more preferably less than about 1% oxygen and wherein the secondary packaging system is selected from the group consisting of a pouch and a blister package.

The present invention is directed to a method of stabilizing an oral pharmaceutical composition comprising:
from about 0.1% to about 5% w/w dronabinol; and
optionally, from about 20% to about 40% w/w water, from about 15% to about 65% w/w of ethanol, from about 2% to about 10% w/w propylene glycol, from about 8% to about 20% w/w polyethylene glycol, and an excipient selected from the group consisting of butylated hydroxyanisole (“BHA”), butylated hydroxytoluene (“BHT”), disodium ethylenediaminetetraacetic acid (“EDTA”), a paraben and a combination thereof,
   comprising the steps of:
a. filling a glass bottle with the oral pharmaceutical composition under a vacuum;
b. capping the glass bottle to create a gaseous headspace in the glass bottle;
c. placing the glass bottle in a secondary packaging system with or without a gas overlay, with or without an oxygen absorbing means; and with or without an oxygen indicator;
d. sealing the secondary packaging system,
   wherein the gaseous headspace contains less than about 20% oxygen, preferably less than about 10% oxygen and more preferably less than about 1% oxygen and wherein the secondary packaging system is selected from the group consisting of a pouch and a blister package.

The present invention is directed to a method of stabilizing an oral pharmaceutical composition comprising:
from about 0.1% to about 5% w/w dronabinol; and
optionally, from about 20% to about 40% w/w water, from about 15% to about 65% w/w of ethanol, from about 2% to about 10% w/w propylene glycol, from about 8% to about 20% w/w polyethylene glycol, and an excipient selected from the group consisting of butylated hydroxyanisole (“BHA”), butylated hydroxytoluene (“BHT”), disodium ethylenediaminetetraacetic acid (“EDTA”), a paraben and a combination thereof,
   comprising the steps of:
a. filling a glass bottle with the oral pharmaceutical composition under a gas overlay;
b. capping the glass bottle to create a gaseous headspace in the glass bottle;
c. placing the glass bottle in a secondary packaging system with or without a gas overlay, with or without an oxygen absorbing means; and with or without an oxygen indicator;
d. sealing the secondary packaging system,
   wherein the gaseous headspace contains less than about 20% oxygen, preferably less than about 10% oxygen and more preferably less than about 1% oxygen and wherein the secondary packaging system is selected from the group consisting of a pouch and a blister package.

The present invention is further directed to a container comprising an oral pharmaceutical composition comprising:
from about 0.1% to about 5% w/w dronabinol; and
optionally, comprising from about 20% to about 40% w/w water, from about 15% to about 65% w/w of ethanol, from about 2% to about 10% w/w propylene glycol, from about 8% to about 20% w/w polyethylene glycol and an excipient selected from the group consisting of butylated hydroxyanisole (BHA), butylated hydroxytoluene (BHT), disodium ethylenediaminetetraacetic acid (EDTA), a paraben and a combination thereof,
   wherein the container further comprises a gaseous headspace containing less than 20% oxygen, preferably less than 10% oxygen and more preferably less than 1% oxygen.

In a preferred embodiment, the gas used in the methods of the present invention is nitrogen or an inert gas. As used herein, the term “inert gas” refers to the gaseous form of an element in which the atoms have a full valence shell. More preferably the inert gas used in the methods of the present invention is selected from the group consisting of helium, neon, argon, krypton, xenon and radon.

In another preferred embodiment, the container is a glass bottle. In a more preferred embodiment, the container is an amber colored glass bottle.

In another preferred embodiment, the container is capped with a screw cap or a crimp cap.

In another preferred embodiment, the secondary packaging system contains an oxygen absorbing means. Preferably, the oxygen absorbing means is provided by one or more walls of the secondary packaging system or by an auxiliary oxygen absorber placed between two or more walls of the secondary packaging system.

In a preferred embodiment, the auxiliary oxygen absorber is an iron based or polymer based oxygen absorber. In a more preferred embodiment, the auxiliary oxygen absorber is an iron based oxygen absorber. In an even more preferred embodiment, the iron based auxiliary oxygen absorber provides absorption of from about 1 to about 3,000 cubic centimeters of oxygen, yet more preferably from about 10 to about 1,000 cubic centimeters of oxygen, even more preferably from about 50 to about 500 cubic centimeters of oxygen and most preferably about 100 cubic centimeters of oxygen.

In another preferred embodiment, the secondary packaging system contains an oxygen indicator.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the compositions of the invention. They are not intended to be limiting in any way.

### EXAMPLES

A preferred composition of the present invention is described in Table 1, below.

TABLE 1
Composition 1

| Ingredients | % w/w | Function |
|---|---|---|
| Dronabinol | 0.541 | Active Ingredient |
| Butylated Hydroxyanisole (BHA) | 0.01 | Anti-oxidant |
| Methylparaben | 0.02 | Preservative |
| Propylparaben | 0.02 | Preservative |
| Sucralose, Micronized | 0.05 | Sweetener |
| Dehydrated ethyl alcohol, 200 proof | 50.0 | Co-solvent |
| Polyethylene glycol 400 | 12.0 | Co-solvent |
| Propylene Glycol | 5.5 | Co-solvent |
| Purified Water | 31.859 | Vehicle |

### Example 1. Preparation of a Composition of the Present Invention

Dronabinol is chemically synthesized as per procedures known to those skilled in the art and is supplied as a clear, amber colored resinous solid at room temperature. A vacuum is applied to dronabinol before heating. Dronabinol is then liquefied by heating in an oven at about 90° C. under vacuum for about sixty to one hundred eighty minutes. Next, the liquefied or molten dronabinol is quickly transferred to a separate stock of dehydrated alcohol and the contents are mixed at 50° C.±5° C., while being sparged with nitrogen in an airtight container until the dronabinol is completely dissolved in dehydrated alcohol to create a 6% w/w dronabinol bulk solution. An excipient solution is then created by dissolving butylated hydroxyl anisole, sucralose, methyl paraben, and propyl paraben in dehydrated alcohol in an air tight tank/container sparged with nitrogen for about fifteen to thirty minutes. Appropriate quantities of PEG 400, propylene glycol, and water are then added while continuing to mix in the air tight tank/container sparged with nitrogen. Next, a calculated amount of dronabinol bulk solution is added to the excipient solution and mixed for about fifteen minutes while continuing to be sparged with nitrogen in an airtight container. Required quantity of dehydrated alcohol is then added and mixed for about ten minutes while the mixture continues to be sparged with nitrogen in an airtight container to give a final aqueous-based oral dronabinol solution containing 0.541% w/w dronabinol as described in Table 1, above.

### Example 2. Bottle Filling Procedure

A composition of the present invention is transferred to an amber-colored glass bottle. The transfer may occur under vacuum or under a gas overlay. More preferably, the gas used to overlay is nitrogen. The bottle is then capped while under a vacuum or a gas overlay (Nitrogen) using either a screw cap or a crimp cap creating a gaseous headspace. The gaseous headspace is then analyzed for oxygen content. Bottles that contain less oxygen than the surrounding atmosphere are sent for secondary packaging.

### Example 3. Packaging Procedure

The amber-colored glass bottles filled with the composition of the present invention are placed in a secondary packaging system under normal atmospheric conditions or under a gas overlay. The secondary packaging system may be comprised of oxygen absorbers or may hold an oxygen absorber. The gas overlay may be provided by a tank placed externally to the packaging. The gas is transferred from the tank to the packaging via a hose, tube or other means at a pressure above 0.01 pounds per square inch (“p.s.i.”), preferably from about 0.1 to about 5 p.s.i.

If an oxygen absorber is introduced in to the packaging system, then the oxygen absorber is placed in the packaging system under a gas overlay.

If an oxygen absorber and/or oxygen indicator are held in the secondary packaging, then the presence of the oxygen absorber and/or oxygen indicator may be assured by visual inspection or an external sensor.

### Example 4. Stability Testing

Secondary packages containing amber-colored glassed bottles filled with a composition of the present invention from Example 3 are subject to stability testing at different storage conditions. The oxygen levels in the gaseous headspace of the bottles are also measured. Briefly, two studies were carried out. In the first study, headspace oxygen was maintained less than 1% and in the second study, the headspace oxygen concentration was less than 10% to assess its effect on stability of the product. Predicted results from these stability tests are seen in Tables 2 through 7.

BQL indicates below quantifiable limit (less than 0.05%)
ND indicates not detected
NP indicates not analyzed

TABLE 2
Headspace Oxygen Levels T₀ (Study 1)

| | Measured Oxygen value |
|---|---|
| Sample Number | in Headspace (%) |
| 1 | 0.561 |
| 2 | 0.231 |
| 3 | 0.903 |
| 4 | 0.361 |
| 5 | 0.193 |
| 6 | 0.360 |
| 7 | 0.139 |
| 8 | 0.574 |
| 9 | 0.402 |
| 10 | 0.622 |
| 11 | 0.673 |
| 12 | 0.088 |
| 13 | 0.377 |
| 14 | 0.515 |
| 15 | 0.241 |
| 16 | 0.483 |
| 17 | 0.660 |
| 18 | 0.589 |
| 19 | 0.652 |
| 20 | 0.684 |
| 21 | 0.208 |
| 22 | 0.728 |
| 23 | 0.510 |
| 24 | 0.27 |
| 25 | 0.385 |
| Average | 0.45 |

Stability data are collected in two studies. In the first study, data is collected at time zero, two weeks, one month, two months and three months at 55° C.; time zero, one month, two months and three months at 40° C.±2° C./75% RH±5% RH; and time zero, one month and three months at 25° C.±2° C./60% RH±5% RH. In the second study, data is collected at time zero, two weeks and one month at 55° C.; and time zero and one month at 40° C.±2° C./75% RH. Assay and impurities are detected using high performance liquid chromatography with an ultraviolet detector at 228 nanometers. Assays are indicated as a percentage of initial concentration and impurities are indicated as a percent area.

TABLE 3
Composition 1 in amber-colored glass bottle
packaged with oxygen absorbers at 25°
C. ± 2° C./60% ± 5% RH

| 25° C. Formulation | RRT | 0 Week | 1 Month | 3 Months |
|---|---|---|---|---|
| Appearance | | Clear | Clear | Clear |
| % O₂ in bottle headspace | | <1 | 0.047 | 0.116 |
| % O₂ in Pouch | | ND | 0 | 0 |
| Assay (% of initial conc.) | | 99.33 | 100.39 | 101.64 |
| Delta-8-THC | 1.20 | 1.72 | 1.67 | 1.64 |
| Cannabinol (CBN) | 0.79 | 0.10 | 0.09 | 0.10 |
| Cannabidiol (CBD) | 0.38 | ND | 0.05 | 0.02 |
| Cis-Delta 9-THC | 0.89 | 0.10 | 0.10 | 0.10 |
| Delta 9-7, 8-Dihydroxy | 0.16 | ND | 0.13 | 0.09 |
| THC | | | | |
| Delta 9-(7 or 8)-Hydroxy | 0.19 | ND | 0.12 | 0.08 |
| THC | | | | |
| Delta 9-7-Hydroxy-8- | 0.25 | ND | 0.07 | 0.07 |
| Ethoxy THC | | | | |
| Delta 6a-8, 10-Dihydroxy | 0.29 | ND | ND | ND |
| THC | | | | |
| Delta 6a, Delta 9-7, | 0.33 | 0.04 | 0.04 | ND |
| 8-Dihydroxy Dihydro- | | | | |
| cannabinol | | | | |
| Delta 9-7-Hydroxy-8- | 0.36 | 0.03 | 0.07 | ND |
| Propoxy THC | | | | |
| Delta 7, Delta 9- | 0.83 | 0.04 | 0.03 | 0.06 |
| Dihydrocannabinol | | | | |
| Delta 8, Delta 10- | 1.15 | ND | ND | 0.02 |
| Dihydrocannabinol | | | | |
| % Unknown Impurities | 0.29 | BQL | 0.05 | BQL |
| | 0.59 | BQL | BQL | 0.05 |
| | 0.65 | BQL | 0.06 | 0.05 |
| | 1.56 | 0.06 | 0.05 | 0.05 |
| % Total Impurities | | 2.09 | 2.53 | 2.33 |

TABLE 4
Composition 1 in amber-colored glass bottle packaged with oxygen absorbers
at 40° C. ± 2° C./75% ± 5% RH

| 40° C. Formulation | RRT | 0 Week | 1 Month | 2 Months | 3 Months |
|---|---|---|---|---|---|
| Appearance | | Clear | Clear | Clear | Clear |
| % O₂ in bottle headspace | | <1 | 0.157 | 0.019 | 0.132 |
| % O₂ in Pouch | | ND | 0 | 0.01 | 0 |
| Assay (% of initial conc.) | | 99.33 | 102.17 | 102.29 | 102.16 |
| Delta-8-THC | 1.20 | 1.72 | 1.64 | 1.64 | 1.63 |
| Cannabinol (CBN) | 0.79 | 0.10 | 0.10 | 0.10 | 0.10 |
| Cannabidiol (CBD) | 0.38 | ND | ND | ND | ND |
| Cis-Delta 9-THC | 0.89 | 0.10 | 0.10 | 0.10 | 0.10 |
| Delta 9-7, 8-Dihydroxy THC | 0.16 | ND | 0.12 | 0.17 | 0.12 |
| Delta 9-(7 or 8)-Hydroxy THC | 0.19 | ND | 0.13 | 0.09 | 0.06 |
| Delta 9-7-Hydroxy-8-Ethoxy THC | 0.25 | ND | 0.08 | 0.10 | 0.08 |
| Delta 6a-8, 10-Dihydroxy THC | 0.29 | ND | ND | ND | ND |
| Delta 6a, Delta 9-7, 8-Dihydroxy | 0.33 | 0.04 | 0.02 | ND | ND |
| Dihydrocannabinol | | | | | |
| Delta 9-7-Hydroxy-8-Propoxy THC | 0.36 | 0.03 | ND | ND | 0.02 |
| Delta 7, Delta 9-Dihydrocannabinol | 0.83 | 0.04 | 0.08 | 0.07 | 0.09 |
| Delta 8, Delta 10- | 1.15 | ND | 0.05 | 0.03 | 0.03 |
| Dihydrocannabinol | | | | | |
| % Unknown Impurities | 0.59 | BQL | 0.09 | 0.12 | 0.10 |
| | 0.65 | BQL | 0.05 | BQL | BQL |
| | 1.56 | 0.06 | 0.05 | BQL | BQL |
| % Total Impurities | | 2.09 | 2.51 | 2.42 | 2.33 |

TABLE 5
Composition 1 in amber-colored glass bottle packaged with oxygen absorbers at 55° C.

| 55° C. Formulation | RRT | 0 Week | 2 Week | 1 Month | 2 Months | 3 Months |
|---|---|---|---|---|---|---|
| Appearance | | Clear | Clear | Clear | Clear | Clear |
| % O₂ in bottle headspace | | <1 | 0 | 0.165 | 0.016 | 0.248 |
| % O₂ in Pouch | | ND | 0.02 | 0 | 0.7 | 0.01 |
| Assay (% of initial conc.) | | 99.33 | 101.76 | 101.37 | 103.86 | 102.78 |
| Delta-8-THC | 1.20 | 1.72 | 1.63 | 1.65 | 1.64 | 1.62 |
| Cannabinol (CBN) | 0.79 | 0.10 | 0.12 | 0.13 | 0.14 | 0.11 |
| Cannabidiol (CBD) | 0.38 | ND | ND | ND | ND | ND |
| Cis-Delta 9-THC | 0.89 | 0.10 | 0.10 | 0.10 | 0.10 | 0.11 |
| Delta 9-7, 8-Dihydroxy THC | 0.16 | ND | 0.24 | 0.21 | 0.23 | 0.12 |
| Delta 9-(7 or 8)-Hydroxy THC | 0.19 | ND | 0.12 | 0.05 | ND | ND |
| Delta 9-7-Hydroxy-8-Ethoxy THC | 0.25 | ND | 0.14 | 0.13 | 0.13 | 0.07 |
| Delta 6a-8, 10-Dihydroxy THC | 0.29 | ND | ND | ND | ND | ND |
| Delta 6a, Delta 9-7, 8-Dihydroxy | 0.33 | 0.04 | 0.02 | ND | 0.04 | 0.02 |
| Dihydrocannabinol | | | | | | |
| Delta 9-7-Hydroxy-8-Propoxy THC | 0.36 | 0.03 | 0.04 | ND | ND | ND |
| Delta 7, Delta 9-Dihydrocannabinol | 0.83 | 0.04 | 0.07 | 0.09 | 0.10 | 0.13 |
| Delta 8, Delta 10- | 1.15 | ND | ND | 0.03 | 0.02 | 0.02 |
| Dihydrocannabinol | | | | | | |
| % Unknown Impurities | 0.59 | BQL | 0.16 | 0.14 | 0.13 | 0.09 |
| | 1.56 | 0.06 | BQL | 0.05 | 0.05 | ND |
| % Total Impurities | | 2.09 | 2.64 | 2.58 | 2.58 | 2.29 |

TABLE 6
Composition 1 in amber-colored glass bottle
packaged with oxygen absorbers at 40°
C. ± 2° C./75% ± 5% RH

| | RRT | 0 Week | 1 Month |
|---|---|---|---|
| Appearance | | Clear | clear |
| % O₂ in bottle headspace | | NP | 8.995 |
| % O₂ in Pouch | | NP | 0.23 |
| Assay (% of initial conc.) | | 99.00 | 101.33 |
| Delta-8-THC | 1.20 | 1.18 | 1.17 |
| Cannabinol (CBN) | 0.79 | 0.21 | 0.18 |
| Cannabidiol (CBD) | 0.38 | ND | 0.04 |
| Cis-Delta 9-THC | 0.89 | 0.11 | 0.11 |
| Delta 9-7, 8-Dihydroxy | 0.17 | BQL | 0.28 |
| THC | | | |
| Delta 9-(7 or 8)-Hydroxy | 0.20 | ND | 0.25 |
| THC | | | |
| Delta 9-7-Hydroxy-8- | 0.26 | ND | 0.16 |
| Ethoxy THC | | | |
| Delta 6a-8, 10-Dihydroxy | 0.30 | ND | ND |
| THC | | | |
| Delta 6a, Delta 9-7, | 0.36 | BQL | 0.07 |
| 8-Dihydroxy Dihydro- | | | |
| cannabinol | | | |
| Delta 9-7-Hydroxy-8- | 0.37 | ND | ND |
| Propoxy THC | | | |
| Delta 7, Delta 9- | 0.83 | 0.04 | ND |
| Dihydrocannabinol | | | |
| Delta 8, Delta 10- | 1.11 | ND | ND |
| Dihydrocannabinol | | | |
| % Unknown Impurities | 0.24 | ND | 0.05 |
| | 0.59 | ND | 0.09 |
| % Total Impurities | | 1.54 | 2.40 |

TABLE 7
Composition 1 in amber-colored glass bottle
packaged with oxygen absorbers at 55° C.

| | RRT | 0 Week | 2 Weeks | 1 Month |
|---|---|---|---|---|
| Appearance | | Clear | clear | |
| % O₂ in bottle headspace | | ND | 6.96 | 5.65 |
| % O₂ in Pouch | | ND | 0.1325 | 0.26 |
| Assay (% of initial conc.) | | 99.00 | 98.90 | 96.89 |
| Delta-8-THC | 1.20 | 1.18 | 1.17 | 1.16 |
| Cannabinol (CBN) | 0.79 | 0.21 | 0.23 | 0.33 |
| Cannabidiol (CBD) | 0.38 | ND | BQL | ND |
| Cis-Delta 9-THC | 0.89 | 0.11 | 0.10 | 0.10 |
| Delta 9-7, 8-Dihydroxy | 0.17 | BQL | 0.59 | 0.95 |
| THC | | | | |
| Delta 9-(7 or 8)-Hydroxy | 0.20 | ND | 0.41 | 0.45 |
| THC | | | | |
| Delta 9-7-Hydroxy-8- | 0.26 | ND | 0.35 | 0.58 |
| Ethoxy THC | | | | |
| Delta 6a-8, 10- | 0.30 | ND | BQL | 0.05 |
| Dihydroxy THC | | | | |
| Delta 6a, Delta 9-7, | 0.36 | BQL | 0.19 | 0.22 |
| 8-Dihydroxy Dihydro- | | | | |
| cannabinol | | | | |
| Delta 9-7-Hydroxy- | 0.37 | ND | ND | ND |
| 8-Propoxy THC | | | | |
| Delta 7, Delta 9- | 0.83 | 0.04 | ND | ND |
| Dihydrocannabinol | | | | |
| Delta 8, Delta 10- | 1.11 | ND | ND | ND |
| Dihydrocannabinol | | | | |
| % Unknown Impurities | 0.29 | ND | 0.05 | BQL |
| | 0.59 | ND | 0.33 | 0.46 |
| % Total Impurities | | 1.54 | 3.42 | 4.30 |

Compositions in amber-colored glass bottles packaged with oxygen absorbers containing less than 1% oxygen in headspace, exhibit less than 3% of total impurities at 25° C.±2° C./60% RH±5% RH, 40° C.±2° C./75% RH±5% RH and 55° C. after 3 months. See, Tables 3-5. Compositions in amber-colored glass bottles packaged with oxygen absorbers containing more than 1% but less than 10% oxygen in headspace, exhibit less than 5% of total impurities at 55° C. See, Table 7. All the individual impurities are within limits as per ICH guidelines at 25° C.±2° C./60% RH±5% RH and 40° C.±2° C./75% RH±5% RH after 3 months for the compositions in bottles with less than 1% oxygen in the headspace. See, Tables 3 and 4.

It is known that dronabinol undergoes oxidative degradation and results in the formation of various impurities when exposed to the atmosphere. Among the oxidative impurities, Delta-9-7, 8-Dihydroxy THC and Delta-9-7-Hydroxy-8-ethoxy THC have significant importance and have tighter FDA specifications. Delta-9-7, 8-Dihydroxy THC in compositions in bottles with less than 1% oxygen in the headspace is present in an amount of 0.12% at 55° C., 0.12% at 40° C.±2° C./75% RH±5% RH and 0.09% at 25° C.±2° C./60% RH±5% RH after 3 months. See, Tables 3, 4 and 5. Compositions in bottles with less than 10% oxygen in the headspace show the same impurity at 0.95% at 55° C. and at 0.28% at 40° C.±2° C./75% RH±5% RH after 1 month. See, Tables 6 and 7. The impurity, Delta-9-7-Hydroxy-8-ethoxy THC in compositions in bottles with less than 1% oxygen in the headspace is present in an amount of 0.07% at 55° C., 0.08% at 40° C.±2° C./75% RH±5% RH and 0.07% at 25° C.±2° C./60% RH±5% RH after 3 months. See, Tables 3, 4 and 5. The same impurity is present in an amount of 0.58% at 55° C. and 0.16% at 40° C.±2° C./75% RH±5% RH after 1 month in compositions in bottles with less than 10% oxygen in the headspace. See, Tables 6 and 7. Thus, the methods of the present invention provide stable dronabinol compositions at room temperature.

What is claimed is:

1. A method of stabilizing an oral pharmaceutical composition comprising from about 0.1% to about 5% w/w dronabinol comprising the steps of:
a. filling a container with the oral pharmaceutical composition under a vacuum or a gas overlay;
b. capping the container to create a gaseous headspace in the container;
c. placing the container in a secondary packaging system under one or more conditions selected from the group consisting of under a gas overlay, with an oxygen absorbing means, and with an oxygen indicator,
d. sealing the secondary packaging system,
wherein the gaseous headspace contains less than 20% oxygen and wherein the secondary packaging system is selected from the group consisting of a pouch and a blister package.

2. The method of claim 1, wherein the gaseous headspace contains less than 10% oxygen.

3. The method of claim 1, wherein the gaseous headspace contains less than 1% oxygen.

4. The method of claim 1, wherein the gas is selected from the group consisting of helium, neon, argon, krypton, xenon, radon and nitrogen.

5. The method of claim 1, wherein the composition further comprises from about 20% to about 40% w/w water, from about 15% to about 65% w/w of ethanol, about 2% to about 10% w/w propylene glycol, about 8% to about 20% w/w polyethylene glycol, and an excipient selected from the group consisting of butylated hydroxyanisole (BHA), butylated hydroxytoluene (BHT), disodium ethylenediaminetetraacetic acid (EDTA), a paraben and a combination thereof.

6. The method of claim 1, wherein container is a glass bottle.

7. The method of claim 6, wherein the glass bottle is amber colored.

8. The method of claim 6, wherein the container is capped with a screw cap or a crimp cap.

9. The method of claim 1, wherein the secondary packaging system contains an oxygen absorbing means.

10. The method of claim 9, wherein the oxygen absorbing means is provided by one or more walls of the secondary packaging system.

11. The method of claim 9, wherein the oxygen absorbing means is provided by an auxiliary oxygen absorber displaced between two or more walls of the secondary packaging system.

12. The method of claim 11, wherein the auxiliary oxygen absorber is in the form of a pellet, strip, sheet, packet or in any other form.

13. The method of claim 11, wherein the auxiliary oxygen absorber is iron based or polymer based.

14. The method of claim 13, wherein the auxiliary oxygen absorber is iron based.

15. The method of claim 13, wherein the iron based auxiliary oxygen absorber provides absorption of from about 1 to about 3,000 cubic centimeters of oxygen.

16. The method of claim 13, wherein the iron based auxiliary oxygen absorber provides absorption of from about 10 to about 1,000 cubic centimeters of oxygen.

17. The method of claim 13, wherein the iron based auxiliary oxygen absorber provides absorption of from about 50 to about 500 cubic centimeters of oxygen.

18. The method of claim 13, wherein the iron based auxiliary oxygen absorber provides absorption of about 100 cubic centimeters of oxygen.

19. The method of claim 1, wherein the secondary packaging system contains an oxygen indicator.

20. A container comprising an oral pharmaceutical composition comprising from about 0.1% to about 5% w/w dronabinol, wherein the container further comprises a gaseous headspace containing less than 20% oxygen.

21. The container of claim 20, wherein the container is a glass bottle.

22. The container of claim 20, wherein the container is an amber colored glass bottle.

23. The container of claim 20, wherein the gaseous headspace contains less than 10% oxygen.

24. The container of claim 20, wherein the gaseous headspace contains less than 1% oxygen.

25. The container of claim 20, further comprising from about 20% to about 40% w/w water, from about 15% to about 65% w/w of ethanol, from about 2% to about 10% w/w propylene glycol and from about 8% to about 20% w/w polyethylene glycol.

26. The container of claim 25, further comprising an excipient selected from the group consisting of butylated hydroxyanisole (BHA), butylated hydroxytoluene (BHT), disodium ethylenediaminetetraacetic acid (EDTA), a paraben and a combination thereof,

27. The container of claim 20, wherein the container comprises a cap.

28. The container of claim 27, wherein the cap is a screw cap or a crimp cap.
